(19) United States
(12) Patent Application Publication
(10) Pub. No.: US 2026/0262919 A1
(43) Pub. Date: Sep. 10, 2026

(54) ENDOSCOPIC IMAGE DIAGNOSIS APPARATUS, ENDOSCOPIC IMAGE DIAGNOSIS METHOD, AND STORAGE MEDIUM

(71) Applicant: NEC Corporation, Tokyo (JP)

(72) Inventor: Shigeaki NAMIKI, Tokyo (JP)

(73) Assignee: NEC Corporation, Tokyo (JP)

(21) Appl. No.: 19/529,552

(22) Filed: Feb. 4, 2026

(30) Foreign Application Priority Data
Mar. 4, 2025 (JP) 2025-033410

(51) Int. Cl. A61B 1/00; G06T 7/00; G16H 50/20

(52) U.S. Cl. CPC A61B 1/000096; A61B 1/00043; G06T 7/0012; G16H 50/20; G06T 2207/10068; G06T 2207/30096; G06T 2207/30168

(57) ABSTRACT

The information processing device 1X includes an acquisition means 31X and an integration means 32X. The acquisition means 31X acquires, for respective types of appearance of endoscopic images used for diagnosis of a subject, identification information on a lesion appearing in each of the endoscopic images, and diagnosis results representing a quality of the lesion based on each of the endoscopic images. The integration means 32X integrates the diagnosis results for each lesion for which the identification information is identical. For example, the information processing device 1X can support decision making based on diagnosis results on endoscopic images.

### INCORPORATION BY REFERENCE

This application is based upon and claims the benefit of priority from Japanese Patent Application No. 2025-033410, filed on Mar. 4, 2025, the disclosure of which is incorporated herein in its entirety by reference.

### TECHNICAL FIELD

The present disclosure relates to a technical field of an information processing apparatus, a method, and a storage medium for processing information on an endoscopic examination.

### BACKGROUND

Conventionally, an image processing system that processes an image obtained by capturing an inside of a lumen of an organ is known. For example, Patent Literature 1 discloses a medical image processing apparatus that recognizes a lesion candidate from a medical image and identifies malignancy of the recognized lesion candidate and an organ included in the medical image.

### Citation List

### Patent Literature

Patent Literature 1: JP 2021-083821 A

### SUMMARY

In general, appearance of an image captured by an endoscope varies depending on a light source or the like used at the time of imaging. Therefore, in a case where qualitative diagnosis of an image obtained by imaging a part suspected of having a lesion with an endoscope is made by computer-aided diagnosis (CAD), it may sometimes be difficult to generate a precise diagnosis result from one image.

In view of the above-described problems, an object of the present disclosure is to provide an information processing apparatus, a method, and a program capable of generating a highly accurate diagnosis result.

In an example aspect of the present disclosure, there is provided an information processing apparatus including: an acquisition means for acquiring, for respective types of appearance of endoscopic images used for diagnosis of a subject, identification information on a lesion appearing in each of the endoscopic images, and diagnosis results representing a quality of the lesion based on each of the endoscopic images; and an integration means for integrating the diagnosis results for each lesion for which the identification information is identical.

In an example aspect of the present disclosure, there is provided a method executed by a computer, including: acquiring, for respective types of appearance of endoscopic images used for diagnosis of a subject, identification information on a lesion appearing in each of the endoscopic images, and diagnosis results representing a quality of the lesion based on each of the endoscopic images; and integrating the diagnosis results for each lesion for which the identification information is identical.

In an example aspect of the present disclosure, there is provided a program executed by a computer, the program causing the computer to: acquire, for respective types of appearance of endoscopic images used for diagnosis of a subject, identification information on a lesion appearing in each of the endoscopic images, and diagnosis results representing a quality of the lesion based on each of the endoscopic images; and integrate the diagnosis results for each lesion for which the identification information is identical.

An example advantage according to the present disclosure is to generate an accurate diagnosis result.

### BRIEF DESCRIPTION OF THE DRAWINGS

FIG. 1 illustrates a schematic configuration of an endoscopy system.

FIG. 2 illustrates an example of a hardware configuration of the image processing apparatus.

FIG. 3 illustrates an example of a data structure of the diagnostic information.

FIG. 4 depicts an example of functional blocks of a processor relating to computer-aided diagnosis.

FIG. 5 depicts an example of functional blocks of the diagnosis unit.

FIG. 6 is a table illustrating a class of the integrated diagnosis result determined in accordance with the first rule information.

FIG. 7 is a table illustrating a class of the integrated diagnosis result determined in accordance with the second rule information.

FIG. 8 is a table illustrating a class of the integrated diagnosis result determined using the third rule information, based on the third integration example.

FIG. 9 illustrates a display screen displayed by the display device in endoscopy.

FIG. 10 depicts an example of a flowchart indicating an outline of processing executed by the image processing apparatus during endoscopy.

FIG. 11 depicts an example of a flowchart indicating an outline of processing executed by the image processing apparatus during endoscopy.

FIG. 12 is a schematic configuration diagram of an endoscopy system.

FIG. 13 is a block diagram of the information processing apparatus.

FIG. 14 illustrates an example of a flowchart indicating a procedure of the process executed by the information processing apparatus.

### EXAMPLE EMBODIMENT

Hereinafter, example embodiments of an information processing device, a method, and a storage medium will be described with reference to the drawings.

### First Example Embodiment

### 1 System Configuration

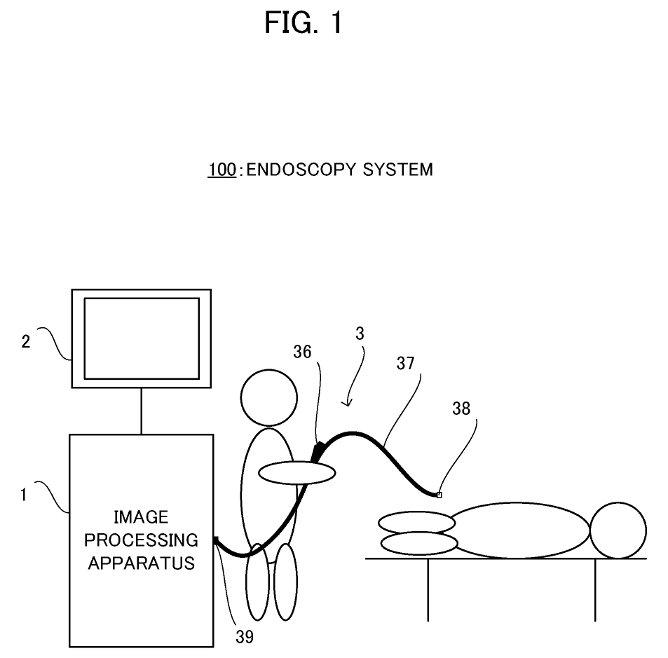

FIG. 1 illustrates a schematic configuration of an endoscopy system 100. The endoscopy system 100 detects a region representing a part of a subject suspected of having a lesion (also referred to as a “lesion region”) from an image captured by an endoscope and makes qualitative diagnosis on the detected lesion region to store and display a diagnosis result. Hereinafter, “qualitative diagnosis” refers to determining quality regarding a detected lesion region, and examples of qualitative diagnosis include classification of a category (diagnosis category) relating to benignancy and malignancy of a lesion, determination of a degree of progression (including depth of invasion and a degree of infiltration), determination of a degree of inflammation, determination of a lesion shape, determination of a lesion size, and the like. The endoscopy system 100 mainly includes an image processing apparatus 1, a display device 2, and an endoscope 3 connected to the image processing apparatus 1 and handled by an examiner such as a doctor who performs endoscopy or treatment.

The image processing apparatus 1 acquires images captured in time series by the endoscope 3 (also referred to as “endoscopic image Ia”) from the endoscope 3 and displays a screen based on the endoscopic images Ia on the display device 2. The endoscopic image Ia is an image captured with a predetermined frame period in at least one of the process of inserting the endoscope 3 into the subject and the process of removing the endoscope 3 from the subject. In the present example embodiment, the image processing apparatus 1 determines the presence or absence of a lesion region, which is a region in an image suspected of a lesion, for each of the time-series endoscopic images Ia. Then, the image processing apparatus 1 performs qualitative diagnosis on the lesion region, using the endoscopic image Ia in which the lesion region has been detected, and stores and displays information based on the generated diagnosis result.

The display device 2 is a display such as a monitor that performs predetermined display based on a display signal supplied from the image processing apparatus 1.

The endoscope 3 mainly includes an operation unit 36 for an examiner to perform predetermined input, a flexible shaft 37 inserted into an organ of a subject to be imaged, a distal end portion 38 incorporating an imaging unit such as an ultra-small imaging element, and a connection unit 39 for connection to the image processing apparatus 1.

The endoscope 3 generates endoscopic images Ia having different types of appearance. For example, the endoscope 3 supports a plurality of types of light sources, and based on input performed via the operation unit 36, the endoscope 3 generates endoscopic images Ia having different types of appearance by switching the light sources used for capturing the endoscopic images Ia. Examples of the light sources include a set of a light source that outputs white light and one or more light sources that output light of a specific wavelength (special light). Note that the generation of endoscopic images Ia having different types of appearance is not limited to switching of the light sources. For example, endoscopic images Ia having different types of appearance may be generated due to at least one of a change in magnification at the imaging unit or a change in a distance between the imaging unit and a subject (lesion region).

The configuration of the endoscopy system 100 illustrated in FIG. 1 is merely an example, and various modifications may be made. For example, the image processing apparatus 1 may be integrated with the display device 2. In another example, the image processing apparatus 1 may be composed of a plurality of apparatuses.

In the present disclosure, a subject of endoscopic examination may be any organ that can be examined by endoscopy, such as the colon, esophagus, stomach, or pancreas. For example, endoscopes to be applied in the present disclosure include a pharyngeal endoscope, a bronchoscope, an upper gastrointestinal endoscope, a duodenoscope, a small-intestinal endoscope, a colonoscope, a capsule endoscope, a thoracoscope, a laparoscope, a cystoscope, a cholangioscope, an arthroscope, a spinal endoscope, an intravascular endoscope, an epiduroscope, and the like. In addition, disease conditions of lesion sites to be detected in endoscopic examination are exemplified as (a) to (f) below.
(a) Head and neck: pharyngeal cancer, malignant lymphoma, papilloma
(b) Esophagus: esophageal cancer, esophagitis, esophageal hiatal hernia, esophageal varices, esophageal achalasia, esophageal submucosal tumor, benign esophageal tumor
(c) Stomach: gastric cancer, gastritis, gastric ulcer, gastric polyp, gastric tumor
(d) Duodenum: duodenal cancer, duodenal ulcer, duodenitis, duodenal tumor, duodenal lymphoma
(e) Small intestine: small-intestinal cancer, small-intestinal neoplastic disease, small-intestinal inflammatory disease, small-intestinal vascular disease
(f) Colon: colon cancer, colonic neoplastic disease, colonic inflammatory disease, colonic polyp, colonic polyposis, Crohn’s disease, colitis, intestinal tuberculosis, hemorrhoids.

### 2 Hardware Configuration

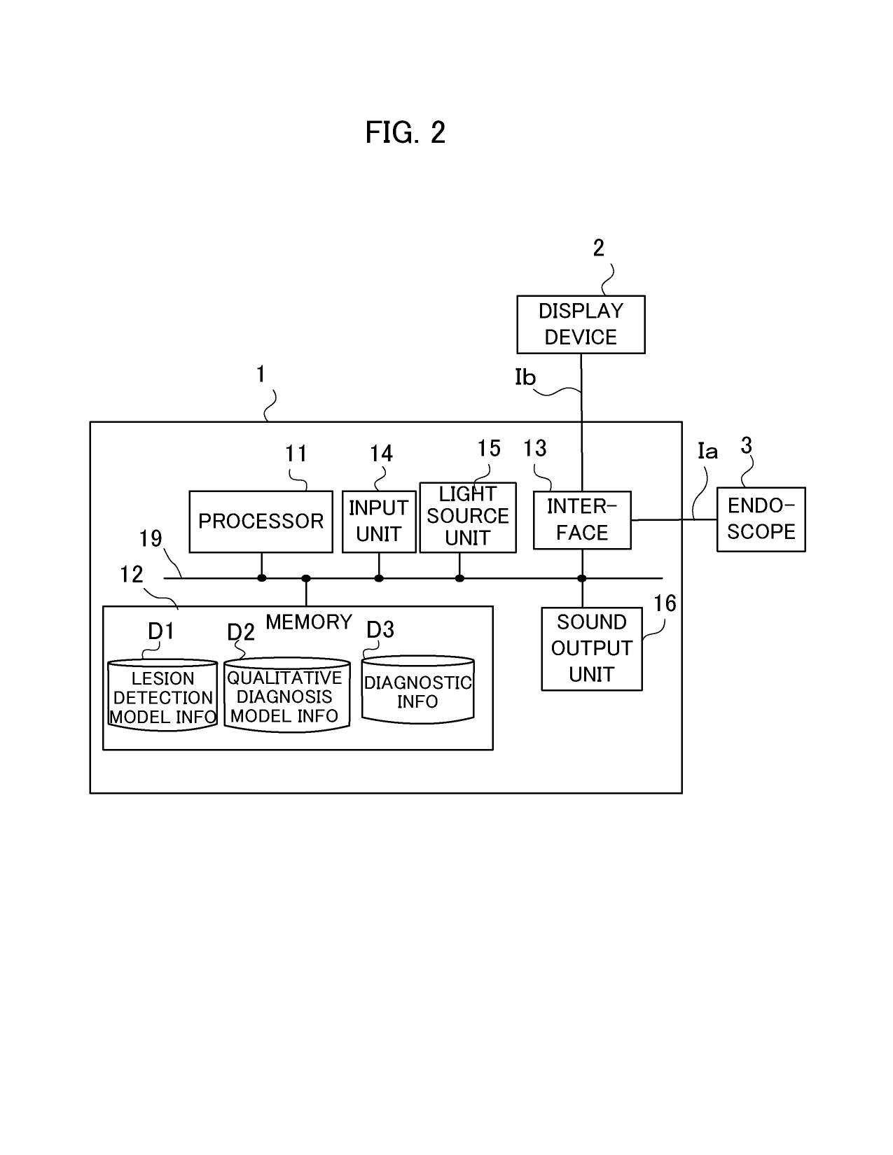

FIG. 2 illustrates a hardware configuration of the image processing apparatus 1. The image processing apparatus 1 mainly includes a processor 11, a memory 12, an interface 13, an input unit 14, a light source unit 15, and a sound output unit 16. These components are connected to each other via a data bus 19.

The processor 11 executes predetermined processing by executing programs and the like stored in the memory 12. The processor 11 is a processor such as a CPU (Central Processing Unit), a GPU (Graphics Processing Unit), or a TPU (Tensor Processing Unit). The processor 11 may be composed of a plurality of processors. The processor 11 is an example of a computer.

The memory 12 includes various types of volatile memories used as working memory, such as a RAM (Random Access Memory) and a ROM (Read Only Memory), and a non-volatile memory that stores information necessary for processing performed by the image processing apparatus 1. The memory 12 may include an external storage device such as a hard disk connected to or incorporated in the image processing apparatus 1, and may include a removable storage medium such as a detachable flash memory. The memory 12 stores programs for causing the image processing apparatus 1 to execute respective processes in the present example embodiment.

The memory 12 also stores lesion detection model information D1, which is information relating to a lesion detection model described later, qualitative diagnosis model information D2, which is information relating to a qualitative diagnosis model described later, and diagnosis information D3, which is information relating to diagnosis results. The memory 12 may further store other information necessary for the image processing apparatus 1 to execute respective processes in the present example embodiment.

The interface 13 performs interface operations between the image processing apparatus 1 and external devices. For example, the interface 13 supplies display information “Ib” generated by the processor 11 to the display device 2. The interface 13 also supplies light generated by the light source unit 15 to the endoscope 3. Further, the interface 13 supplies the processor 11 with an electrical signal indicating the endoscopic image Ia supplied from the endoscope 3. The interface 13 may be a communication interface such as a network adapter for performing wired or wireless communication with external devices, or may be a hardware interface compliant with standards such as USB (Universal Serial Bus) or SATA (Serial AT Attachment).

The input unit 14 generates an input signal based on an operation performed by an examiner. The input unit 14 includes, for example, buttons, a touch panel, a remote controller, or a voice input device. The sound output unit 16 outputs sound based on control by the processor 11.

The light source unit 15 generates light to be supplied to the distal end portion 38 of the endoscope 3. The light source unit 15 may also incorporate a pump or the like for feeding water or air to be supplied to the endoscope 3. For example, the light source unit 15 includes a plurality of types of switchable light sources. In this case, the light source unit 15 controls light emission of the light sources such that a light source designated based on input information supplied from the endoscope 3 or the input unit 14 emits light. Note that the light source unit 15 may be incorporated in the endoscope 3 instead of being included in the image processing apparatus 1.

### 3 Machine Learning Model

Next, details of a lesion detection model and a qualitative diagnosis model will be described.

The lesion detection model is a machine learning model that generates a detection result regarding a lesion intended to be detected in endoscopy, and parameters necessary for the lesion detection model are stored in lesion detection model information D1. For example, in a case where an endoscopic image is input, the lesion detection model outputs a detection result on a particular lesion (also referred to as a “lesion detection result”) in the input endoscopic image. In other words, the lesion detection model is a model that has learned the relationship between the image input into the lesion detection model and the presence or absence of a particular lesion in the input image. The lesion detection model may be a model (including a statistical model, the same applies hereinafter) including any architecture adopted in machine learning, such as a neural network or a support vector machine. Examples of a representative model of such a neural network include Fully Convolutional Network, SegNet, U-Net, V-Net, Feature Pyramid Network, Mask R-CNN, and DeepLab. In a case where the lesion detection model is constituted by the neural network, the lesion detection model information D1 includes, for example, various parameters such as a layer structure, a neuron structure of each layer, the number of filters and a filter size in each layer, and a weight of each element of each filter.

The lesion detection result output by the lesion detection model may be, for example, information representing a certainty factor that exists for each piece of identification information (lesion identifier (ID)) on a lesion intended to be detected, or may be information indicating a lesion ID of which the certainty factor is equal to or more than a predetermined threshold value. The lesion detection result may also include information indicating a range of a lesion region (for example, bounding box information) existing in the input endoscopic image. The lesion detection result may be a map indicating a certainty factor as to being a lesion region, for each unit region of the input endoscopic image. The above-mentioned map is an image indicating the reliability as to being a lesion region for each pixel or pixel block and may be a mask image indicating the lesion region by binary. The lesion detection model may be a model prepared for each lesion ID and designed to separately output a lesion detection result for its relevant lesion ID. In this case, a machine-learned parameter of the lesion detection model for each lesion ID is included in the lesion detection model information D1.

The lesion detection model is trained in advance based on a set of an input image conforming to an input format of the lesion detection model and ground truth data indicating a correct answer of an inference result supposed to be output by the lesion detection model in a case where the input image is input. Then, parameters and the like of each model obtained by training are stored in a memory 12 as the lesion detection model information D1.

The qualitative diagnosis model is a machine learning model that infers (classifies) the quality regarding a lesion region, and parameters necessary for the qualitative diagnosis model are stored in qualitative diagnosis model information D2. For example, in a case where an image indicating a lesion region is input, the qualitative diagnosis model outputs an inference result indicating the quality of the lesion indicated by the image that has been input. In other words, the qualitative diagnosis model is a model that has learned the relationship between the image input into the qualitative diagnosis model and the quality of a lesion in the input image. The image input into the qualitative diagnosis model may be, for example, an image obtained by cutting out a lesion region from an endoscopic image, or may be the entire endoscopic image including a lesion region. The inference result indicating the quality of the lesion indicates, for example, a certainty factor for each category (diagnosis category) regarding benignancy and malignancy of the lesion. The inference result indicating the quality of the lesion is not limited to the information regarding the diagnosis category and may indicate a certainty factor for each degree of progression (including depth of invasion and a degree of infiltration). In addition, the inference result indicating the quality of the lesion may indicate a certainty factor for each degree of inflammation, or may be information regarding the size or shape of the lesion (for example, information indicating a relationship between the size or shape and its certainty factor). In this manner, the inference result indicating the quality of the lesion includes, for example, a candidate class and the certainty factor of each class.

The qualitative diagnosis model may be a model (including a statistical model, the same applies hereinafter) including any architecture adopted in machine learning, such as a neural network or a support vector machine. In a case where the qualitative diagnosis model is constituted by the neural network, the qualitative diagnosis model information D2 includes, for example, various parameters such as a layer structure, a neuron structure of each layer, the number of filters and a filter size in each layer, and a weight of each element of each filter. The qualitative diagnosis model is trained in advance based on a set of an input image conforming to an input format of the qualitative diagnosis model and ground truth data indicating a correct answer of an inference result supposed to be output by the qualitative diagnosis model in a case where the input image is input. Then, parameters and the like of each model obtained by training are stored in the memory 12 as the qualitative diagnosis model information D2.

In the qualitative diagnosis model, machine learning may be performed for each type of appearance of the endoscopic image Ia, and machine-learned parameters may be stored in the qualitative diagnosis model information D2. In this case, parameters of the qualitative diagnosis models used for each type of appearance of the endoscopic image Ia are separately included in the qualitative diagnosis model information D2. For example, in a case where a light source is switched in a light source unit 15, a qualitative diagnosis model used to infer the quality of a lesion region in the related endoscopic image Ia is associated with a light source for each kind of light sources.

### 4 Diagnostic Information

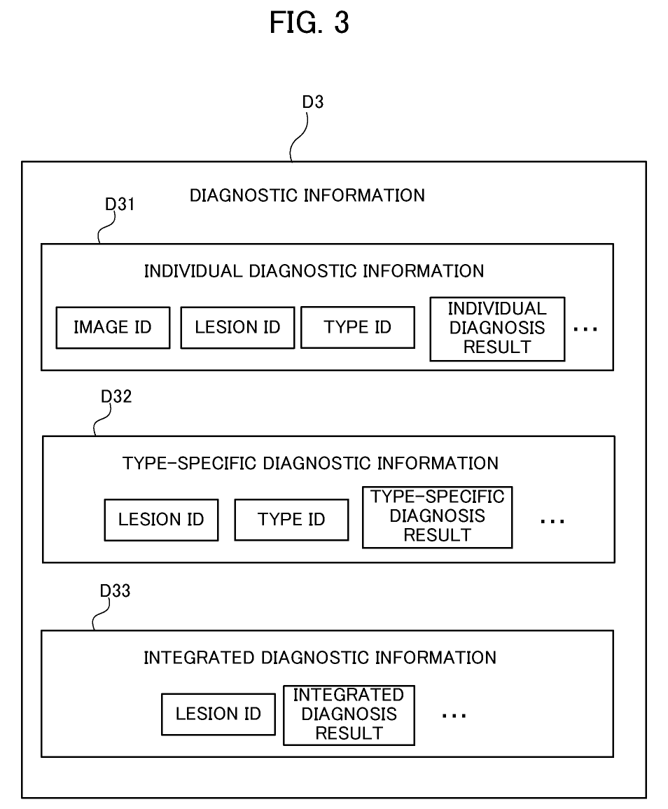

Diagnostic information D3 is information indicating a qualitative diagnosis result generated by the image processing apparatus 1 using the qualitative diagnosis model. FIG. 3 illustrates an example of a data structure of the diagnostic information D3. The diagnostic information D3 illustrated in FIG. 3 includes individual diagnostic information D31, type-specific diagnostic information D32, and integrated diagnostic information D33.

The individual diagnostic information D31 is diagnostic information for each endoscopic image Ia on which the qualitative diagnosis using the qualitative diagnosis model has been made and includes a record generated for each endoscopic image Ia on which the qualitative diagnosis has been made. Each record of the individual diagnostic information D31 includes an “image ID”, a “lesion ID”, a “type ID”, and an “individual diagnosis result”.

The “image ID” denotes identification information on the endoscopic image Ia on which the qualitative diagnosis has been made. The “lesion ID” denotes identification information on a lesion detected in the endoscopic image Ia on which the qualitative diagnosis has been made. The “type ID” denotes identification information on the type of appearance of the endoscopic image Ia on which the qualitative diagnosis has been made. For example, in a case where the light source is switched in the light source unit 15, the “type ID” may denote identification information on a light source (light source ID) used to capture the endoscopic image Ia. In another example, the “type ID” may denote distinct identification information uniquely defined based on at least one of the light source ID, a zoom magnification at the time of imaging, or a degree of perspective (a close view or a distant view) of a captured lesion region.

The “individual diagnosis result” denotes a qualitative diagnosis result on a lesion region by the qualitative diagnosis model. For example, the “individual diagnosis result” includes a candidate class and a certainty factor of each class. Specifically, in a case where the qualitative diagnosis model generates an inference result regarding the diagnosis category, the “individual diagnosis result” indicates a certainty factor as to existence for each diagnosis category. In addition, the “individual diagnosis result” may indicate a certainty factor for each degree of inflammation, or may denote information regarding the size or shape of a lesion (for example, information indicating a relationship between the size or shape and the certainty factor).

Each record of the individual diagnostic information D31 may further include condition information indicating the condition of the endoscopic image Ia, and the like, in addition to each sort of information described above.

The type-specific diagnostic information D32 denotes diagnostic information obtained by integrating the individual diagnostic information D31 for each combination of the lesion ID and the type ID. Each record of the type-specific diagnostic information D32 includes a “lesion ID”, a “type ID”, and a “type-specific diagnosis result”. The “lesion ID” and the “type ID” indicate relevant lesion ID and type ID, respectively. The “type-specific diagnosis result” indicates a diagnosis result obtained by integrating individual diagnosis results in the individual diagnostic information D31 for each relevant lesion ID and type ID.

The integrated diagnostic information D33 denotes diagnostic information obtained by integrating the type-specific diagnostic information D32 for each lesion ID. Each record of the integrated diagnostic information D33 includes a “lesion ID” and an “integrated diagnosis result”. The “lesion ID” indicates a relevant lesion ID. The “integrated diagnosis result” indicates a diagnosis result obtained by integrating the type-specific diagnosis results in the type-specific diagnostic information D32 having different type IDs, for each relevant lesion ID. Details of this integration method will be described later.

### 5 Functional Block

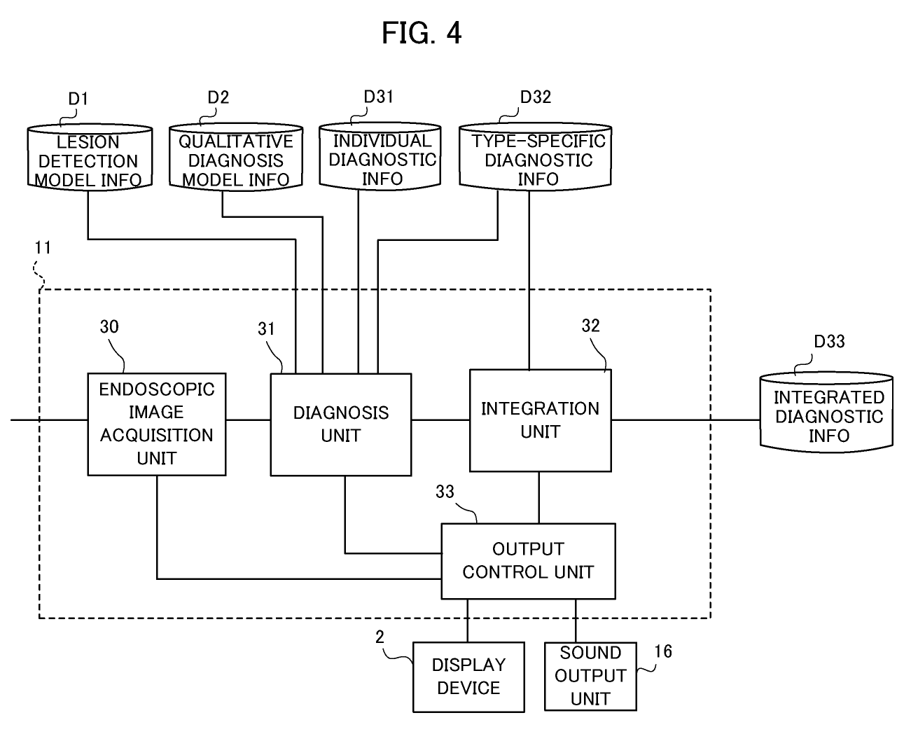

FIG. 4 depicts an example of functional blocks of a processor 11 relating to computer-aided diagnosis. The processor 11 of the image processing apparatus 1 functionally includes an endoscopic image acquisition unit 30, a diagnosis unit 31, an integration unit 32, and an output control unit 33. While blocks that exchange data with each other are connected by a solid line in FIG. 4, a combination of blocks that exchange data with each other is not limited to this. The same applies to other diagrams of functional blocks described later.

The endoscopic image acquisition unit 30 acquires the endoscopic images Ia captured by the endoscope 3 via an interface 13 at a predetermined interval. The image ID is assigned to each endoscopic image Ia. Then, the endoscopic image acquisition unit 30 supplies the acquired endoscopic image Ia to each of the diagnosis unit 31 and the output control unit 33. Each processing unit in a subsequent stage then repeatedly performs processing described later with a time interval at which the endoscopic image acquisition unit 30 acquires the endoscopic image Ia, as one cycle.

The diagnosis unit 31 performs diagnosis on the endoscopic image Ia supplied from the endoscopic image acquisition unit 30, based on the lesion detection model information D1 and the qualitative diagnosis model information D2, and generates the individual diagnostic information D31 and the type-specific diagnostic information D32, based on the generated diagnosis result. Detailed processing of the diagnosis unit 31 will be described later.

The integration unit 32 generates the integrated diagnostic information D33 obtained by integrating the type-specific diagnostic information D32 generated for each type of appearance. For example, in a case where the diagnosis unit 31 generates records of the type-specific diagnostic information D32 having different type IDs for a certain lesion ID, the integration unit 32 generates a record of the diagnostic information D3 obtained by integrating the records having different type IDs. For example, in a case where the endoscopic images Ia are captured by switching between a first light source and a second light source, the integration unit 32 first generates, for a certain lesion ID, a first record of the type-specific diagnostic information D32 having a type ID relevant to the first light source and a second record of the type-specific diagnostic information D32 having a type ID relevant to the second light source. Next, the integration unit 32 generates a record of the diagnostic information D3 obtained by integrating the first record and the second record.

The output control unit 33 generates display information Ib, based on the latest endoscopic image Ia supplied from the endoscopic image acquisition unit 30, the individual diagnostic information D31 and the type-specific diagnostic information D32 generated by the diagnosis unit 31, and the integrated diagnostic information D33 generated by the integration unit 32. Then, the output control unit 33 supplies the generated display information Ib to the display device 2 to display the latest endoscopic image Ia, the diagnosis result, and the like on the display device 2. The output control unit 33 may perform sound output control of a sound output unit 16 in such a way as to output a warning sound, voice guidance, or the like for notifying a user of detection of a lesion and a qualitative diagnosis result regarding the lesion, based on the diagnosis result.

Here, each component of the endoscopic image acquisition unit 30, the diagnosis unit 31, the integration unit 32, and the output control unit 33 can be implemented by, for example, the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like.

### 6 Diagnosis Unit

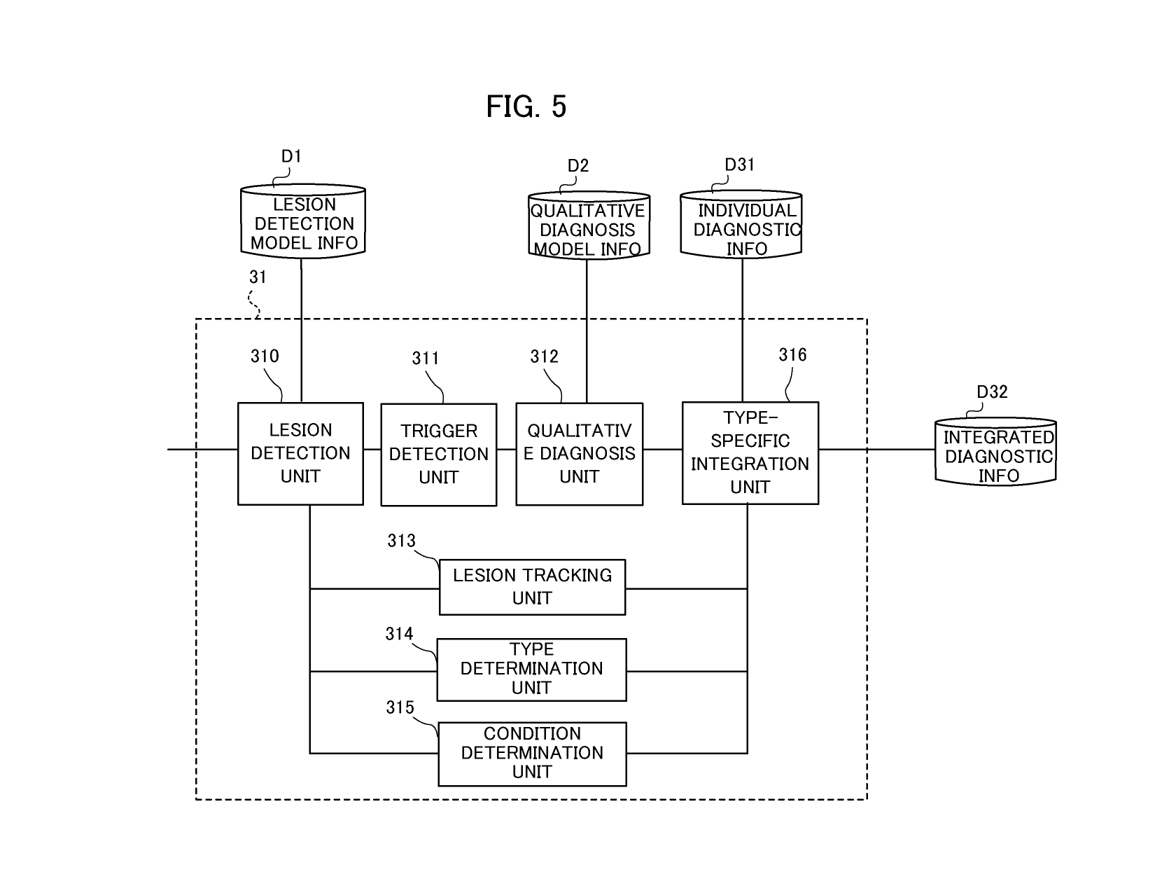

FIG. 5 depicts an example of functional blocks of the diagnosis unit 31. The diagnosis unit 31 functionally includes a lesion detection unit 310, a trigger detection unit 311, a qualitative diagnosis unit 312, a lesion tracking unit 313, a type determination unit 314, a condition determination unit 315, and a type-specific integration unit 316.

The lesion detection unit 310 acquires a lesion detection result output by the lesion detection model configured with reference to the lesion detection model information D1, by inputting the endoscopic image Ia into the lesion detection model. The lesion detection unit 310 supplies the generated lesion detection result and the endoscopic image Ia to the trigger detection unit 311, the lesion tracking unit 313, the type determination unit 314, the condition determination unit 315, and the like. In this case, the lesion detection unit 310 may supply the generated lesion detection result and the endoscopic image Ia only in a case where it is determined based on the generated lesion detection result that a lesion exists. In a case where the lesion detection model is a model machine-trained for each lesion ID, the lesion detection unit 310 acquires lesion detection results obtained by inputting the endoscopic image Ia into each of the lesion detection models for each lesion ID and associates the acquired lesion detection result with the lesion ID relevant to the used lesion detection model.

In a case where a lesion detection result indicating that a lesion has been detected is supplied from the lesion detection unit 310, the trigger detection unit 311 detects a predetermined input from the examiner serving as a trigger for qualitative diagnosis through an input unit 14 or an operation unit 36. Then, in a case where the predetermined input from the examiner serving as a trigger for qualitative diagnosis has been detected by the input unit 14 or the operation unit 36, the trigger detection unit 311 determines that there has been a trigger for making qualitative diagnosis and supplies the lesion detection result and the endoscopic image Ia to the qualitative diagnosis unit 312.

The qualitative diagnosis unit 312 makes qualitative diagnosis, based on the endoscopic image Ia and the lesion detection result on the endoscopic image Ia when the trigger detection unit 311 determines that there has been a trigger for making qualitative diagnosis, and the qualitative diagnosis model configured with the qualitative diagnosis model information D2. In this case, for example, the qualitative diagnosis unit 312 first identifies a lesion region from the endoscopic image Ia, based on the lesion detection result and the like, and generates a cropped image obtained by cutting out the identified lesion region from the endoscopic image Ia. Then, the qualitative diagnosis unit 312 acquires an inference result output by the qualitative diagnosis model by inputting the cropped image of the lesion region into the qualitative diagnosis model. The qualitative diagnosis unit 312 then treats the acquired inference result as an individual diagnosis result and associates the image ID of the endoscopic image Ia used for qualitative diagnosis with the individual diagnosis result. Then, the qualitative diagnosis unit 312 supplies the image ID and the individual diagnosis result to the type-specific integration unit 316. In a case where the qualitative diagnosis model has been machine-trained for each type of appearance, the qualitative diagnosis unit 312 selects a qualitative diagnosis model according to the type ID determined by the type determination unit 314. In this case, the qualitative diagnosis unit 312 generates an individual diagnosis result, based on the selected qualitative diagnosis model and the endoscopic image Ia having the image ID associated with the type ID.

The lesion tracking unit 313 tracks a lesion in the time-series endoscopic images Ia, based on the endoscopic images Ia and the lesion detection result supplied from the lesion detection unit 310. For example, the lesion tracking unit 313 assigns a unique lesion ID to a lesion appearing in common in the time-series endoscopic images Ia, based on a classification result on the lesion indicated by the lesion detection result and/or information indicating the lesion region (for example, bounding box information). In this case, the lesion tracking unit 313 may track the lesion, using any tracking technique. Thus, the lesion tracking unit 313 associates the lesion ID with the image ID of the endoscopic image Ia in which the lesion has been detected. Thereafter, the lesion tracking unit 313 supplies, for example, the image ID and the lesion ID to the type-specific integration unit 316.

The type determination unit 314 determines (identifies) the type of appearance of the endoscopic image Ia supplied from the lesion detection unit 310, based on the supplied endoscopic image Ia and the like. In this case, the type determination unit 314 may determine the type of appearance by image analysis on the endoscopic image Ia, or may determine the type of appearance, based on a signal supplied from the outside. The type determination unit 314 associates the type ID that identifies the type of the appearance of the endoscopic image Ia with the image ID of that endoscopic image Ia and supplies the image ID and the type ID to the type-specific integration unit 316.

Here, a specific example of a method for determining the type of appearance will be described. For example, in a case where the endoscopic image Ia is captured by switching between a plurality of kinds of light sources, the type determination unit 314 identifies the light source ID of the light source used at the time of capturing the endoscopic image Ia, based on a signal or the like supplied from the light source unit 15. Then, the type determination unit 314 determines the type ID according to the identified light source ID. In another example, the type determination unit 314 acquires information regarding the zoom magnification of an imaging unit (endoscope lens) (for example, which of a magnifying endoscope or a normal endoscope is used) from the endoscope 3 and determines the type ID according to the identified magnification from the acquired information. In still another example, the type determination unit 314 conducts image analysis on a lesion region identified according to the endoscopic image Ia and the lesion detection result to determine whether the lesion region is in a distant view or a close view (that is, a degree of perspective) and determines the type ID according to the determination result. The type determination unit 314 may identify the type ID from a combination of the kind of the light source, the zoom magnification, and the degree of perspective described above. In this case, information indicating relevant type IDs is stored in the memory 12 or the like for each combination candidate of the kind of the light source, the zoom magnification, and the degree of perspective, and the type determination unit 314 identifies the type ID with reference to the stored information.

The type determination unit 314 may also determine the type ID, using a machine learning model on which machine learning has been performed beforehand. In this case, in the machine learning model mentioned above, machine learning has been performed in such a way that, in a case where an image is input, an inference result regarding the type ID of the image that has been input is output, and machine-trained parameters are stored in the memory 12. Then, the type determination unit 314 determines the type ID, based on the inference result output by the machine learning model in response to an input of the endoscopic image Ia or the cropped image of the lesion region into the machine learning model.

The condition determination unit 315 determines the condition of the endoscopic image Ia supplied from the lesion detection unit 310. In this case, the condition determination unit 315 may determine the condition level of the endoscopic image Ia, using any condition determination technique or using a machine learning model on which machine learning has been performed beforehand. Then, the condition determination unit 315 supplies condition information representing the condition of the endoscopic image Ia and the image ID of that endoscopic image Ia to the type-specific integration unit 316.

The type-specific integration unit 316 generates a record of the individual diagnostic information D31 including the image ID and the individual diagnosis result supplied from the qualitative diagnosis unit 312. At this time, the type-specific integration unit 316 identifies the lesion ID associated with the image ID in the generated record, based on the image ID and the lesion ID supplied from the lesion tracking unit 313, and includes the identified lesion ID in the record. Similarly, the type-specific integration unit 316 refers to the image ID and the type ID supplied from the type determination unit 314, and the image ID and the condition information supplied from the condition determination unit 315, and includes the type ID and the condition information associated with the image ID of the generated record, in the record. In this manner, the type-specific integration unit 316 associates the individual diagnosis result, the lesion ID, the type ID, and the condition information for the common image ID and registers the associated information as a record of the individual diagnostic information D31.

The type-specific integration unit 316 integrates the records registered in the individual diagnostic information D31 for each type ID and registers a record representing the integrated information in the type-specific diagnostic information D32. For example, in a case where the type ID or the lesion ID supplied from the type determination unit 314 has changed, the type-specific integration unit 316 extracts the record of the individual diagnostic information D31 associated with the lesion ID and the type ID immediately before the change. Then, the type-specific integration unit 316 generates a record of the type-specific diagnostic information D32 from the extracted record of the individual diagnostic information D31. In this case, the type-specific integration unit 316 generates a record of the type-specific diagnostic information D32 in which the lesion ID and the type ID immediately before the change are associated with a type-specific diagnosis result obtained by integrating the individual diagnosis result included in the extracted record of the individual diagnostic information D31 for each type ID.

Here, the integration of the individual diagnosis results will be supplementarily described. For example, in a case where the individual diagnosis result represents a candidate class and a relevant certainty factor (that is, a score), the type-specific integration unit 316 computes an average value (or another statistical representative value such as a median value) of the certainty factors for each class, based on the individual diagnosis results included in a plurality of extracted records of the individual diagnostic information D31. Then, the type-specific integration unit 316 includes the type-specific diagnosis result representing the average value or the like of the certainty factors for each class in the record of the type-specific diagnostic information D32.

At this time, the type-specific integration unit 316 may set a weight for each of the individual diagnosis results, based on the condition information, and perform weighted averaging. In this case, the type-specific integration unit 316 sets a higher weight as the condition level indicated by the condition information is higher and computes a weighted average of the certainty factors for each class candidate. In this case, for example, information (formula, lookup table, or the like) indicating the relationship between the condition level and the weight supposed to be set is stored in the memory 12 or the like, and the type-specific integration unit 316 refers to the stored information and sets the weight of each individual diagnosis result according to the condition information. The type-specific integration unit 316 may also perform processing such as excluding an individual diagnosis result regarded as an outlier from integration targets, based on any statistical approach.

### 7 Integration Unit

Next, specific examples (a first integration example to a third integration example) in which records of the type-specific diagnostic information D32 having different type IDs are integrated to generate a record of the integrated diagnostic information D33 will be described. Hereinafter, specific examples of generating an integrated diagnosis result from the type-specific diagnosis results associated with different type IDs will be described. The type-specific diagnosis result indicates a candidate class (such as a diagnosis category) and a certainty factor of each candidate class.

In the first integration example, the integration unit 32 finds an average (may be other statistical representative values such as a median value, the same applies hereinafter) of certainty factors of each class indicated by the type-specific diagnosis results for each type ID. Then, the integration unit 32 compares the averages of the certainty factors for each class and generates an integrated diagnosis result indicating a class having the highest average of the certainty factors. For example, in a case of integrating N type-specific diagnosis results indicating the certainty factors for each of M classes, the integration unit 32 computes an average of the certainty factors of the N type-specific diagnosis results for each of the M classes. Then, the integration unit 32 identifies a class indicating the highest average among the averages of the certainty factors of the M classes and generates an integrated diagnosis result indicating the identified class. The type-specific integration unit 316 may perform processing such as excluding an individual diagnosis result regarded as an outlier from integration targets, based on any statistical approach.

In the second integration example, first, the integration unit 32 refers to a relevant type-specific diagnosis result for each type ID and identifies a class having the highest certainty factor. Hereinafter, the class having the highest certainty factor for each type ID will be also referred to as a “maximum likelihood class”. Then, the integration unit 32 selects one class from the maximum likelihood classes for each type ID in accordance with rule information stored in the memory 12 or the like and generates an integrated diagnosis result indicating the selected class.

Here, first rule information and second rule information that are specific examples of the rule information will be described.

The first rule information is information indicating the priority of each class (that is, a priority order list for the classes). In a case where there are two different classes as a result of aggregating the maximum likelihood classes for each type ID, the integration unit 32 generates an integrated diagnosis result indicating the maximum likelihood class having a higher rank in the priority order indicated by the first rule information.

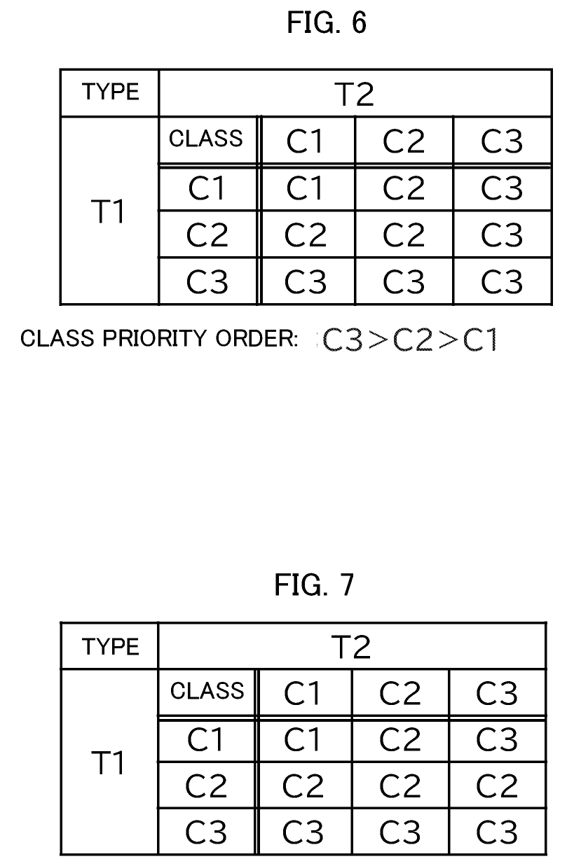

FIG. 6 is a table illustrating a class of the integrated diagnosis result determined in accordance with the first rule information. Here, there are two kinds of type IDs of “T1” and “T2”, and the candidate classes are three classes of “C1” to “C3”. Then, the priority order between the classes is defined to be “C3 > C2 > C1” by the first rule information.

The table illustrated in FIG. 6 indicates a class preferentially selected as the integrated diagnosis result for each combination of the maximum likelihood classes of the type IDs “T1” and “T2”. For example, in a case where the maximum likelihood class of the type ID “T1” is “C3” and the maximum likelihood class of the type ID “T2” is “C1”, the integration unit 32 generates an integrated diagnosis result indicating “C3” in accordance with the priority order “C3 > C2 > C1” between the classes. In another example, in a case where the maximum likelihood class of the type ID “T1” is “C1” and the maximum likelihood class of the type ID “T2” is “C2”, the integration unit 32 generates an integrated diagnosis result indicating “C2” in accordance with the priority order “C3 > C2 > C1” between the classes.

The second rule information is information (reference table) indicating a prioritized class for each combination of the maximum likelihood classes between the type IDs. For example, the rule information of the second example is information indicating, for a certain type ID, a class for which the maximum likelihood class of the certain type ID is prioritized.

FIG. 7 is a table illustrating a class of the integrated diagnosis result determined in accordance with the second rule information.

In FIG. 7, when the maximum likelihood class of the type ID “T1” is “C2” or “C3”, the maximum likelihood class of the type ID “T1” is adopted as the integrated diagnosis result. For example, when the maximum likelihood class of the type ID “T1” is “C2” and the maximum likelihood class of the type ID “T2” is “C3”, “C2” is adopted as the integrated diagnosis result.

On the other hand, when the maximum likelihood class of the type ID “T2” is “C1”, the maximum likelihood class of the type ID “T1” is adopted as the integrated diagnosis result. For example, when the maximum likelihood class of the type ID “T1” is “C1” and the maximum likelihood class of the type ID “T2” is “C3”, “C3” is adopted as the integrated diagnosis result.

The third integration example corresponds to a combination of the first integration example and the second integration example, and the integration unit 32 determines the integrated diagnosis result in accordance with the rule information. The rule information in the third integration example (also referred to as “third rule information”) indicates which one of the first integration example and the second integration example is to be executed, according to the combination of the maximum likelihood classes for each type ID. Furthermore, the third rule information includes information indicating a class to be prioritized for a combination of the maximum likelihood classes for each type ID designated that the second integration example is to be executed.

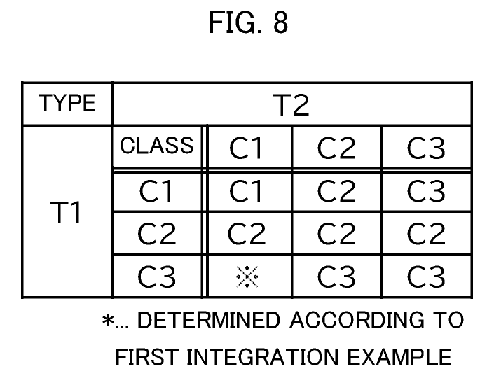

FIG. 8 is a table illustrating a class of the integrated diagnosis result determined using the third rule information, based on the third integration example.

The third rule information indicates that, in a case of a combination of “C3” as the maximum likelihood class of the type ID “T1” and “C1” as the maximum likelihood class of the type ID “T2”, the class of the integrated diagnosis result is determined according to the first integration example. That is, in this case, the integration unit 32 finds an average of the certainty factors of each class indicated by the type-specific diagnosis results for each type ID and generates an integrated diagnosis result indicating the class having the highest average of the certainty factors. The third rule information defines that, when the maximum likelihood class of the type ID “T1” is “C2” or “C3” other than the above combination, the maximum likelihood class of the type ID “T1” is preferentially used as the integrated diagnosis result. For example, when the maximum likelihood class of the type ID “T1” is “C2” and the maximum likelihood class of the type ID “T2” is “C3”, “C2” is adopted as the integrated diagnosis result. On the other hand, the third rule information defines that, when the maximum likelihood class of the type ID “T2” is “C1”, the maximum likelihood class of the type ID “T1” is preferentially used as the integrated diagnosis result. For example, when the maximum likelihood class of the type ID “T1” is “C1” and the maximum likelihood class of the type ID “T2” is “C3”, “C3” is adopted as the integrated diagnosis result.

### 8 Display Example

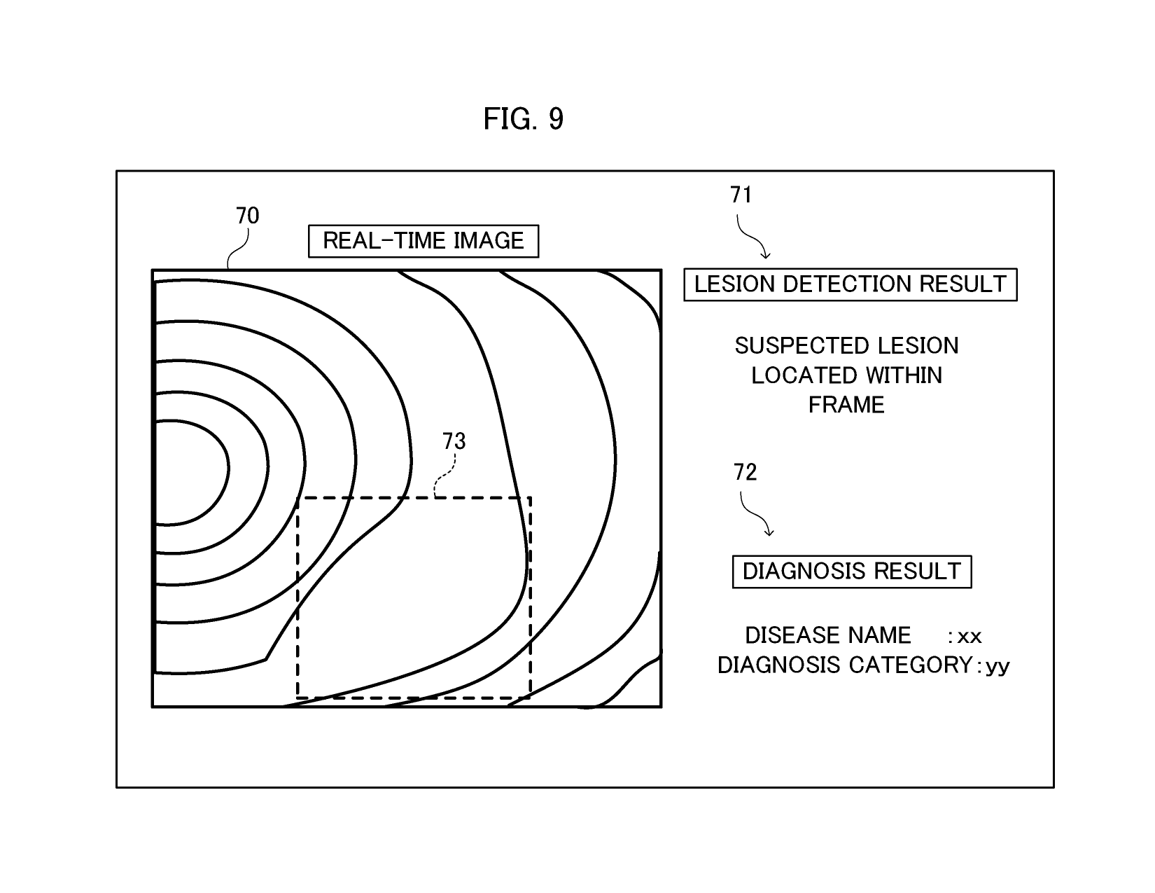

FIG. 9 illustrates a display screen displayed by the display device 2 in endoscopy. The output control unit 33 of the image processing apparatus 1 outputs, to the display device 2, the display information Ib generated based on the latest endoscopic image Ia supplied from the endoscopic image acquisition unit 30 and the lesion detection result and the diagnosis result generated by the diagnosis unit 31. The output control unit 33 displays the display screen described above on the display device 2 by transmitting the display information Ib to the display device 2.

In FIG. 9, the output control unit 33 of the image processing apparatus 1 provides a real-time image display area 70, a lesion detection result display area 71, and a diagnosis result display area 72 on the display screen.

Here, the output control unit 33 displays a moving image representing the latest endoscopic image Ia in the real-time image display area 70. Furthermore, in the lesion detection result display area 71, the output control unit 33 displays information based on the lesion detection result generated by the diagnosis unit 31. Since the lesion detection result indicating that a lesion region has been detected based on the endoscopic image Ia displayed in the real-time image display area 70 has been obtained, the output control unit 33 displays text information “suspected lesion located within frame” indicating that a lesion exists, in the lesion detection result display area 71. Here, since information on the bounding box that identifies the lesion region is included in the lesion detection result, the output control unit 33 superimposes a broken line frame 73 corresponding to the above bounding box onto the latest endoscopic image Ia in the real-time image display area 70.

The output control unit 33 may output, through the sound output unit 16, a sound (including voice) notifying that a lesion is highly likely to exist.

Furthermore, the output control unit 33 displays information based on the integrated diagnostic information D33 generated by the diagnosis unit 31 in the diagnosis result display area 72. Here, the latest record of the integrated diagnostic information D33 is referred to, and the disease name (“xx”) relevant to the lesion ID included in that latest record and the diagnosis category (“yy”) indicated by the integrated diagnosis result are displayed in the diagnosis result display area 72. The output control unit 33 may output, by voice, the disease name and the diagnosis category through the sound output unit 16.

In the display example illustrated in FIG. 9, the output control unit 33 can present a highly accurate diagnosis result or the like to the examiner in real time.

### 9 Processing Flow

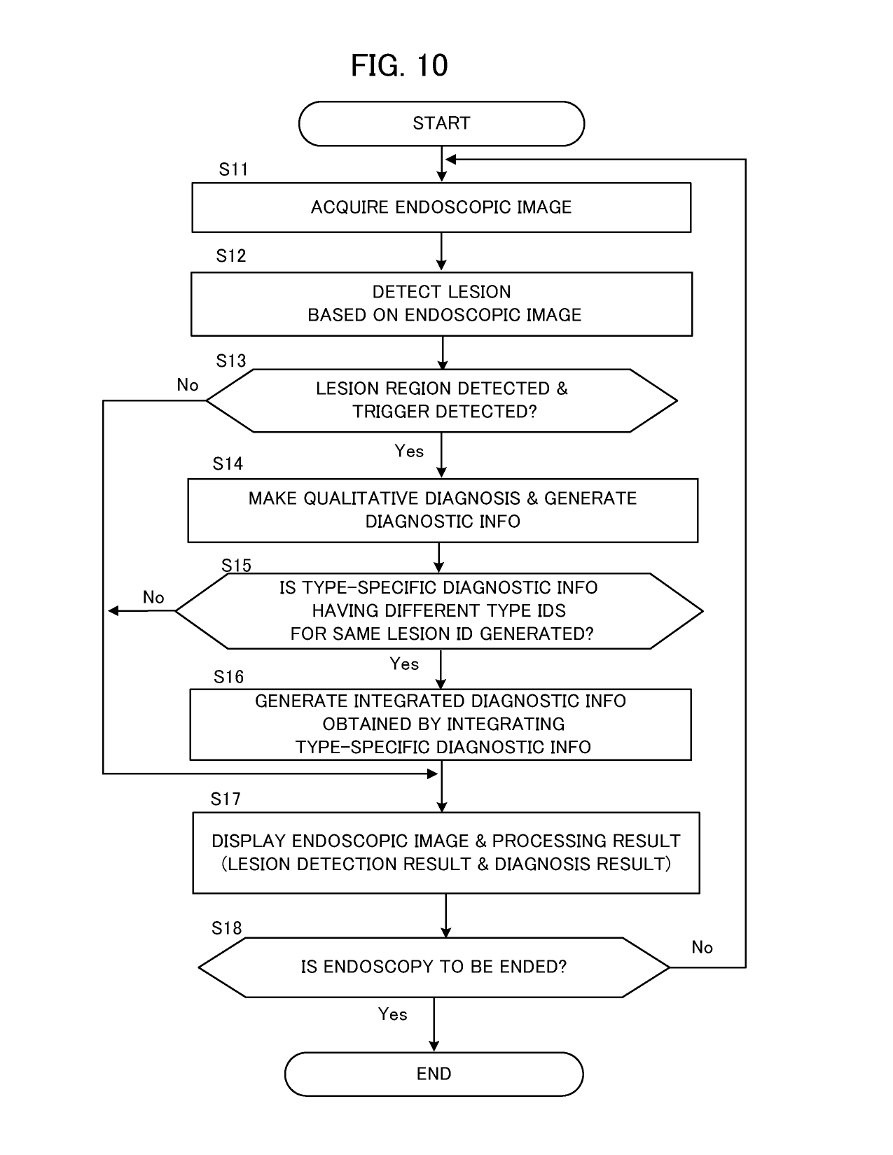

FIG. 10 depicts an example of a flowchart indicating an outline of processing executed by the image processing apparatus 1 during endoscopy.

First, the image processing apparatus 1 acquires the endoscopic image Ia (step S11). In this case, the endoscopic image acquisition unit 30 of the image processing apparatus 1 receives the endoscopic image Ia from the endoscope 3 via the interface 13.

Next, the image processing apparatus 1 detects a lesion in the endoscopic image Ia acquired in step S11 (step S12). In this case, the image processing apparatus 1 acquires the lesion detection result output from the lesion detection model by inputting the endoscopic image Ia into the lesion detection model configured with reference to the lesion detection model information D1.

Then, the image processing apparatus 1 determines whether a lesion region has been detected from the endoscopic image Ia and a trigger for qualitative diagnosis based on an operation by the examiner has been detected (step S13). Then, in a case where the image processing apparatus 1 has determined that a lesion region has been detected from the endoscopic image Ia and a trigger for qualitative diagnosis has been detected (step S13; Yes), the image processing apparatus 1 makes qualitative diagnosis and generates the diagnostic information D3 (step S14). In this case, the image processing apparatus 1 generates a record of the individual diagnostic information D31 including the individual diagnosis results for each endoscopic image Ia on which the qualitative diagnosis has been performed. The image processing apparatus 1 also generates a record of the type-specific diagnostic information D32 for each type ID obtained by integrating the records of the individual diagnostic information D31, for example, at a timing when the type ID is switched.

Meanwhile, in a case where no lesion region has been detected from the endoscopic image Ia, or in a case where no trigger for qualitative diagnosis has been detected (step S13; No), the processing proceeds to step S17.

After step S14, the image processing apparatus 1 determines whether records of the type-specific diagnostic information D32 having different type IDs for the same lesion ID have been generated (step S15). Then, in a case where the image processing apparatus 1 determines that the type-specific diagnostic information D32 having the same lesion ID but different type IDs has been generated (step S15; Yes), the image processing apparatus 1 generates a record of the integrated diagnostic information D33 obtained by integrating the records of the type-specific diagnostic information D32 (step S16). On the other hand, in a case where no type-specific diagnostic information D32 having the same lesion ID but different type IDs has been generated (step S15; No), the processing proceeds to step S17.

Then, in step S17, the image processing apparatus 1 displays, on the display device 2, information based on the endoscopic image Ia obtained in step S11, the lesion detection result generated in step S12, and the diagnostic information D3 generated in steps S14 and S16 (step S17). In a case where the image processing apparatus 1 determines, in step S13, that no lesion region has been recognized, the image processing apparatus 1 may display the endoscopic image Ia and information indicating that no lesion region has been recognized, in step S17.

Then, the image processing apparatus 1 determines whether the endoscopy has ended (step S18). For example, in a case where the image processing apparatus 1 has detected a predetermined input or the like into the input unit 14 or the operation unit 36, the image processing apparatus 1 determines that the endoscopy has ended. Then, in a case where the image processing apparatus 1 determines that the endoscopy has ended (step S18; Yes), the image processing apparatus 1 ends the processing in the flowchart. On the other hand, in a case where the image processing apparatus 1 determines that the endoscopy has not ended (step S18; No), the image processing apparatus 1 returns the processing to step S11.

### 10 Modification

Next, modifications suitable for the above-described first example embodiment will be described. The following modifications may be combined and applied to the above-described first example embodiment.

### First Modification

In the flowchart in FIG. 10, the image processing apparatus 1 generates a record of the integrated diagnostic information D33 including the integrated diagnosis result in step S16 in a case where the image processing apparatus 1 determines, in step S15, that the type-specific diagnostic information D32 having different types has been generated. Alternatively to this, in a case where the image processing apparatus 1 determines that the integrated diagnosis result can be confirmed from the type-specific diagnosis results of a particular type in accordance with the rule information, the image processing apparatus 1 may generate a record of the integrated diagnostic information D33 including the integrated diagnosis result even if no type-specific diagnosis result of other types has been obtained.

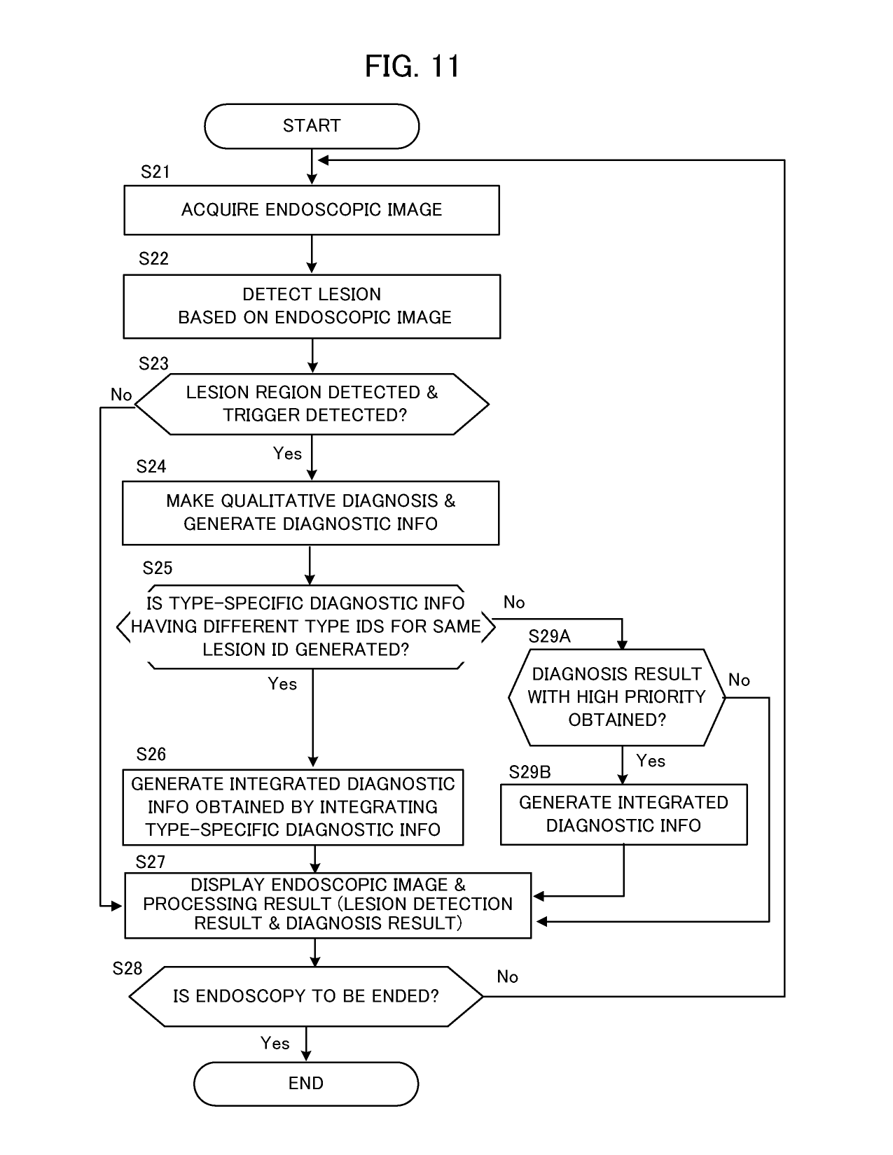

FIG. 11 depicts an example of a flowchart indicating an outline of processing executed by the image processing apparatus 1 during endoscopy. Since steps S21 to S26 are the same as steps S11 to S16, the description thereof will be omitted as appropriate.

In a case where the image processing apparatus 1 determines, in step S25, that no type-specific diagnostic information D32 having the same lesion ID but different type IDs has been generated (step S25; No), the image processing apparatus 1 determines whether a type-specific diagnosis result with high priority has already been obtained (step S29A). For example, in a case of using the third rule information for generating the integrated diagnosis result illustrated in FIG. 8, if a type-specific diagnosis result in which the maximum likelihood class is “C2” for the type ID “T1” is obtained, the integrated diagnosis result has “C2” regardless of the content of the type-specific diagnosis result of the type ID “T2”. Accordingly, in such a case, the image processing apparatus 1 determines, in step S29A, that a type-specific diagnosis result that can confirm the integrated diagnosis result has been obtained, regardless of other type-specific diagnosis results.

Then, in a case where a type-specific diagnosis result with high priority has already been obtained (step S29A; Yes), the image processing apparatus 1 determines that the integrated diagnosis result can be confirmed. Therefore, in this case, the image processing apparatus 1 generates a record of the integrated diagnostic information D33 including the integrated diagnosis result determined based on the already generated type-specific diagnosis result (step S29B). On the other hand, in a case where no type-specific diagnosis result with high priority has been obtained (step S29A; No), the image processing apparatus 1 determines that the integrated diagnosis result cannot be confirmed and advances the processing to step S27. In step S27, the image processing apparatus 1 displays, on the display device 2, information based on the endoscopic image Ia obtained in step S21, the lesion detection result generated in step S12, and the diagnostic information D3 generated in steps S24 and S26 or step S29B (step S27). Then, the image processing apparatus 1 determines whether the endoscopy has ended (step S28). Then, in a case where the image processing apparatus 1 determines that the endoscopy has ended (step S28; Yes), the image processing apparatus 1 ends the processing in the flowchart. On the other hand, in a case where the image processing apparatus 1 determines that the endoscopy has not ended (step S28; No), the image processing apparatus 1 returns the processing to step S11.

According to the present modification, the image processing apparatus 1 can quickly confirm the integrated diagnosis result and notify the examiner of the integrated diagnosis result.

### Second Modification

The image processing apparatus 1 may process a video made up of the endoscopic images Ia generated at the time of endoscopy, after the endoscopy.

For example, in a case where a video to be subjected to processing is designated based on a user input or the like through the input unit 14 at any timing after endoscopy, the image processing apparatus 1 sequentially performs the processing in the flowchart in FIG. 10 or 11 on the time-series endoscopic images Ia constituting the designated video. Then, in a case where the image processing apparatus 1 determines, in step S18 or step S28, that the video subjected to processing has ended, the image processing apparatus 1 ends the processing in the flowchart and returns to step S11 in a case where the video subjected to processing has not ended, while performing the processing in the flowchart by focusing on the next endoscopic image Ia in time series.

### Third Modification

Some functions of the image processing apparatus 1 may be implemented by a storage device different from the image processing apparatus 1.

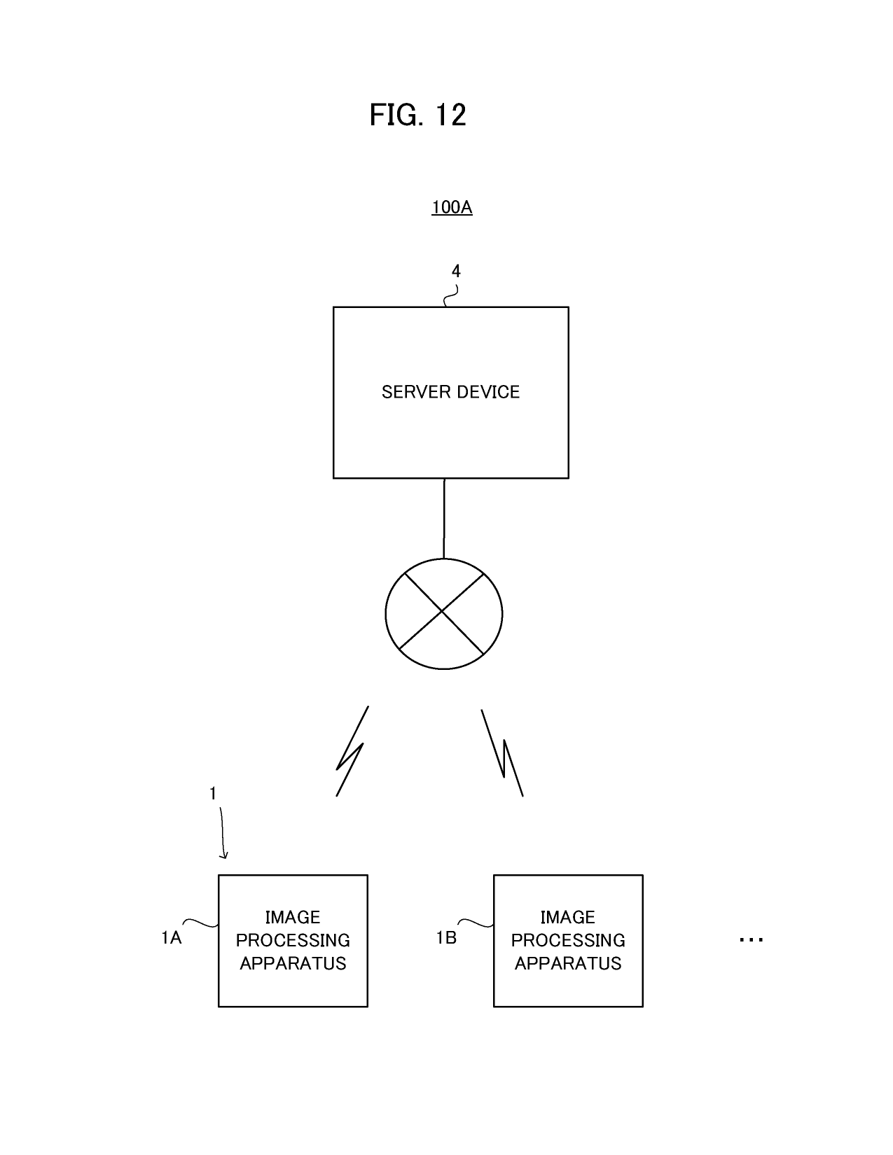

FIG. 12 is a schematic configuration diagram of an endoscopy system 100A according to a modification. Note that, for the sake of simplicity, the display device 2, the endoscope 3, and the like are not illustrated. The endoscopy system 100A includes a server device 4 and a plurality of image processing apparatuses 1 (1A, 1B,...) capable of performing data communication with the server device 4 via a network.

Each image processing apparatus 1 performs data communication with the server device 4 via a network. The interface 13 of each image processing apparatus 1 includes a communication interface such as a network adapter for performing communication. In this configuration, the server device 4 may instead execute at least a part of the processing executed by each functional block of the processor 11 of the image processing apparatus 1 illustrated in FIG. 4. The server device 4 may hold at least one of the lesion detection model information D1, the qualitative diagnosis model information D2, and the diagnostic information D3 instead of each image processing apparatus 1.

### Second Example Embodiment

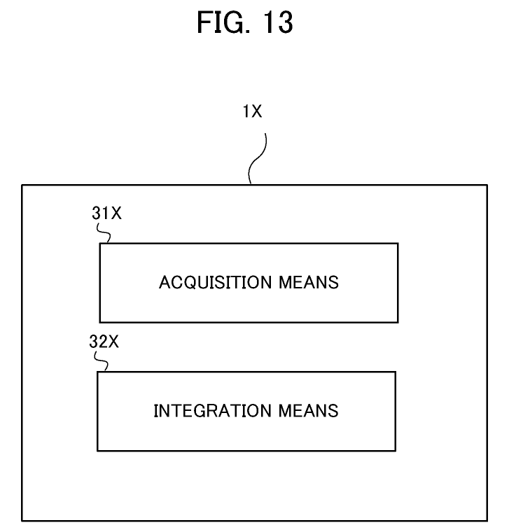

FIG. 13 is a block diagram of the information processing apparatus 1X. The information processing apparatus 1X includes an acquisition means 31X and an integration means 32X. The information processing apparatus 1X may be configured by plural devices.

The acquisition means 31X is configured to acquire, for respective types of appearance of endoscopic images used for diagnosis of a subject, identification information on a lesion appearing in each of the endoscopic images, and diagnosis results representing a quality of the lesion based on each of the endoscopic images. Examples of the acquisition means 31X include the diagnosis unit 31 according to the first example embodiment.

The integration means 32X is configured to integrate the diagnosis results for each lesion for which the identification information is identical. Examples of the integration means 32X include the integration unit 32 according to the first example embodiment.

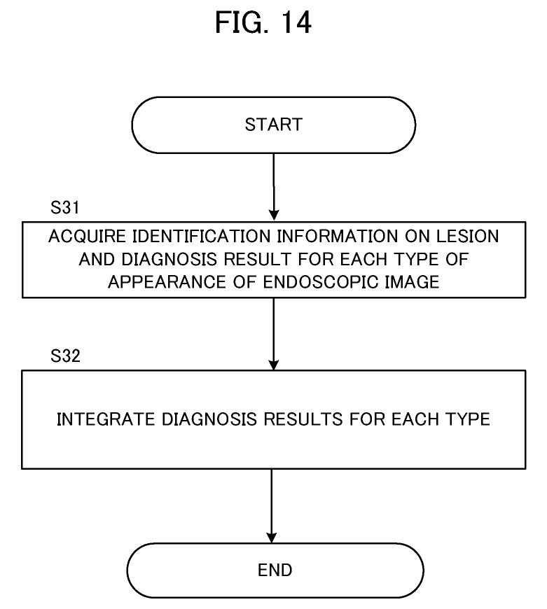

FIG. 14 illustrates an example of a flowchart indicating a procedure of the process executed by the information processing apparatus 1X. The acquisition means 31X acquires, for respective types of appearance of endoscopic images used for diagnosis of a subject, identification information on a lesion appearing in each of the endoscopic images, and diagnosis results representing a quality of the lesion based on each of the endoscopic images (step S31). The integration means 32X integrates the diagnosis results for each lesion for which the identification information is identical (step S32).

The information processing device 1X according to the second example embodiment can generates an accurate diagnosis result obtained by integrating qualitative diagnosis results for respective appearance types.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

In addition, part or all of the above-described example embodiments (including modifications thereof; the same applies hereinafter) may also be described as the following Supplementary Notes, but the present disclosure is not limited thereto. Further, part or all of the configurations described in the Supplementary Notes dependent on Supplementary Note 1 may also depend on Supplementary Notes 9 and 10 in the same dependent manner as the Supplementary Notes dependent on Supplementary Note 1. Furthermore, without being limited to the apparatuses, methods, and storage media described in the Supplementary Notes, part or all of the configurations described as the Supplementary Notes may similarly be applied, without departing from the scope of the above-described example embodiments, to methods, various types of hardware, software, various recording media (including storage media) for recording software, or systems.

### Supplementary Note 1

An information processing apparatus comprising:
an acquisition means for acquiring, for respective types of appearance of endoscopic images used for diagnosis of a subject,
identification information on a lesion appearing in each of the endoscopic images, and
diagnosis results representing a quality of the lesion based on each of the endoscopic images; and
an integration means for integrating the diagnosis results for each lesion for which the identification information is identical.

### Supplementary Note 2

The information processing apparatus according to Supplementary Note 1, further comprising a determination means for
identifying a kind of a light source used to capture each of the endoscopic images, and
determining the respective types of appearance, based on the identified kind of the light source.

### Supplementary Note 3

The information processing apparatus according to Supplementary Note 1, further comprising a determination means
for identifying at least one of perspective appearance of the lesion or magnification of each of the endoscopic images, and
determining the respective types, based on at least one of the identified perspective appearance or magnification.

### Supplementary Note 4

The information processing apparatus according to Supplementary Note 1, wherein
the diagnosis result indicates a class to which the quality belongs and a score for each class, and
the integration means computes, based on the diagnosis results for the respective types, a representative value of the scores for each class, and
generates an integrated diagnosis based on the representative value of each class.

### Supplementary Note 5

The information processing apparatus according to Supplementary Note 1, wherein
the diagnosis result indicates a class to which the quality belongs, and
the integration means generates an integrated diagnosis result from the diagnosis results for the respective types in accordance with rule information indicating a priority among the classes.

### Supplementary Note 6

The information processing apparatus according to Supplementary Note 1, wherein
the diagnosis result indicates classes to which the quality belongs, and
the integration means generates an integrated diagnosis result from the diagnosis results for the respective types in accordance with rule information indicating a prioritized class among combinations of the classes indicated by the diagnosis results for the respective types.

### Supplementary Note 7

The information processing apparatus according to Supplementary Note 1, further comprising:
a determination means for determining a condition of each of the endoscopic images, wherein
upon detecting a plurality of the diagnosis results having an identical type, the acquisition means acquires the diagnosis results for the respective types, by integrating the plurality of the diagnosis results, based on a determination result on the conditions of the endoscopic images used for the plurality of the diagnosis results.

### Supplementary Note 8

The information processing apparatus according to Supplementary Note 1, further comprising an output control means for outputting, through a display device or a voice output device, a diagnosis result obtained by integrating the diagnosis results for the respective types.

### Supplementary Note 9

A method executed by a computer, comprising:
acquiring, for respective types of appearance of endoscopic images used for diagnosis of a subject,
identification information on a lesion appearing in each of the endoscopic images, and
diagnosis results representing a quality of the lesion based on each of the endoscopic images; and
integrating the diagnosis results for each lesion for which the identification information is identical.

### Supplementary Note 10

A non-transitory computer readable storage medium storing a program executed by a computer, the program causing the computer to:
acquiring, for respective types of appearance of endoscopic images used for diagnosis of a subject,
identification information on a lesion appearing in each of the endoscopic images, and
diagnosis results representing a quality of the lesion based on each of the endoscopic images; and
integrating the diagnosis results for each lesion for which the identification information is identical.

### Supplementary Note 11

A non-transitory computer readable storage medium storing the program according to Supplementary Note 10.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. Each example embodiment can be appropriately combined with other example embodiments. All Patent and Non-Patent Literatures mentioned in this specification are incorporated by reference in its entirety.

### DESCRIPTION OF REFERENCE NUMERALS

1, 1A, 1B Image processing apparatus

2 Display device

3 Endoscope

11 Processor

12 Memory

13 Interface

14 Input unit

15 Light source unit

16 Sound output unit

100, 100A Endoscopy system

What is claimed is:

1. An endoscopic image diagnosis apparatus comprising:
at least one memory configured to store instructions, and
at least one processor configured to execute the instructions to:
acquire, for respective types of appearance of endoscopic images used for diagnosis of a subject,
identification information on a lesion appearing in each of the endoscopic images, and
diagnosis results representing a quality of the lesion based on each of the endoscopic images; and
integrate the diagnosis results for each lesion for which the identification information is identical.

2. The endoscopic image diagnosis apparatus according to claim 1, wherein the at least one processor is configured to further execute the instructions to
identify a kind of a light source used to capture each of the endoscopic images, and
determine the respective types of appearance, based on the identified kind of the light source.

3. The endoscopic image diagnosis apparatus according to claim 1, wherein the at least one processor is configured to further execute the instructions to
identify at least one of perspective appearance of the lesion or magnification of each of the endoscopic images, and
determine the respective types, based on at least one of the identified perspective appearance or magnification.

4. The endoscopic image diagnosis apparatus according to claim 1, wherein
the diagnosis result indicates a class to which the quality belongs and a score for each class, and
the at least one processor is configured to execute the instructions to
compute, based on the diagnosis results for the respective types, a representative value of the scores for each class, and
generate an integrated diagnosis based on the representative value of each class.

5. The endoscopic image diagnosis apparatus according to claim 1, wherein
the diagnosis result indicates a class to which the quality belongs, and
the at least one processor is configured to execute the instructions to generate an integrated diagnosis result from the diagnosis results for the respective types in accordance with rule information indicating a priority among the classes.

6. The endoscopic image diagnosis apparatus according to claim 1, wherein
the diagnosis result indicates classes to which the quality belongs, and
the at least one processor is configured to execute the instructions to generate an integrated diagnosis result from the diagnosis results for the respective types in accordance with rule information indicating a prioritized class among combinations of the classes indicated by the diagnosis results for the respective types.

7. The endoscopic image diagnosis apparatus according to claim 1, wherein the at least one processor is configured to further execute the instructions to
determine a condition of each of the endoscopic images, and
upon detecting a plurality of the diagnosis results having an identical type, acquire the diagnosis results for the respective types, by integrating the plurality of the diagnosis results, based on a determination result on the conditions of the endoscopic images used for the plurality of the diagnosis results.

8. The endoscopic image diagnosis apparatus according to claim 1, wherein the at least one processor is configured to further execute the instructions to display or output as audio a diagnosis result obtained by integrating the diagnosis results for the respective types.

9. The endoscopic image diagnosis apparatus according to claim 1, wherein the at least one processor is configured to further execute the instructions to input the endoscopic images into a qualitative diagnosis model and acquire the diagnosis results output by the qualitative diagnosis, wherein the qualitative diagnosis model is trained, through machine learning, to learn a relationship between an endoscopic image including a lesion region and a quality of a lesion in the endoscopic image.

10. An endoscopic image diagnosis method executed by a computer, comprising:
acquiring, for respective types of appearance of endoscopic images used for diagnosis of a subject,
identification information on a lesion appearing in each of the endoscopic images, and
diagnosis results representing a quality of the lesion based on each of the endoscopic images; and
integrating the diagnosis results for each lesion for which the identification information is identical.

11. The endoscopic image diagnosis method according to claim 10, further comprising
identifying a kind of a light source used to capture each of the endoscopic images, and
determine the respective types of appearance, based on the identified kind of the light source.

12. The endoscopic image diagnosis method according to claim 10, further comprising
identifying at least one of perspective appearance of the lesion or magnification of each of the endoscopic images, and
determining the respective types, based on at least one of the identified perspective appearance or magnification.

13. The endoscopic image diagnosis method according to claim 10, wherein
the diagnosis result indicates a class to which the quality belongs and a score for each class, and
the integrating the diagnosis results comprises
computing, based on the diagnosis results for the respective types, a representative value of the scores for each class, and
generating an integrated diagnosis based on the representative value of each class.

14. The endoscopic image diagnosis method according to claim 10, wherein
the diagnosis result indicates a class to which the quality belongs, and
the integrating the diagnosis results comprises generating an integrated diagnosis result from the diagnosis results for the respective types in accordance with rule information indicating a priority among the classes.

15. The endoscopic image diagnosis method according to claim 10, wherein
the diagnosis result indicates classes to which the quality belongs, and
the integrating the diagnosis results comprises generating an integrated diagnosis result from the diagnosis results for the respective types in accordance with rule information indicating a prioritized class among combinations of the classes indicated by the diagnosis results for the respective types.

16. The endoscopic image diagnosis method according to claim 10, further comprising
determining a condition of each of the endoscopic images, and
upon detecting a plurality of the diagnosis results having an identical type, acquire the diagnosis results for the respective types, by integrating the plurality of the diagnosis results, based on a determination result on the conditions of the endoscopic images used for the plurality of the diagnosis results.

17. The endoscopic image diagnosis method according to claim 10, further comprising
displaying a diagnosis result obtained by integrating the diagnosis results for the respective types.

18. The endoscopic image diagnosis method according to claim 10, further comprising
outputting as audio a diagnosis result obtained by integrating the diagnosis results for the respective types.

19. The endoscopic image diagnosis method according to claim 10, further comprising
inputting the endoscopic images into a qualitative diagnosis model and acquiring the diagnosis results output by the qualitative diagnosis,
wherein the qualitative diagnosis model is trained, through machine learning, to learn a relationship between an endoscopic image including a lesion region and a quality of a lesion in the endoscopic image.

20. A non-transitory computer readable storage medium storing a program executed by a computer, the program causing the computer to:
acquiring, for respective types of appearance of endoscopic images used for diagnosis of a subject,
identification information on a lesion appearing in each of the endoscopic images, and
diagnosis results representing a quality of the lesion based on each of the endoscopic images; and
integrating the diagnosis results for each lesion for which the identification information is identical.
